(19) United States
(12) Patent Application Publication
(10) Pub. No.: US 2003/0119882 A1
(43) Pub. Date: Jun. 26, 2003

(54) Solid pharmaceutical composition containing torasemide

(76) Inventor: Markus Maegerlein, Mennheim (DE)

Correspondence Address: Herbert B. Keil, KEIL & WEINKAUF, 1350 Connecticut Ave., N.W., Washington, DC, 20036, US

(21) Appl. No.: 10/271,976

(22) Filed: Oct. 17, 2002

(30) Foreign Application Priority Data
Oct. 22, 2001 (DE) 101 51 973.7

(51) Int. Cl. A61K 31/44; A61K 9/20

(52) U.S. Cl. 514/353; 424/465

(57) ABSTRACT

The present invention relates to a solid pharmaceutical composition, comprising

a) torasemide of the modification 11 or a solvate thereof and

b) at least 50% by weight, based on the total weight of the composition, of at least one excipient which is selected from a sugar, sugar alcohol, calcium hydrogenphosphate, cellulose, cellulose derivatives and polyvinylpyrrolidones having a molecular weight in the range from 1000 to 15 000.

The pharmaceutical composition according to the invention has an active compound release profile which is comparable to that of corresponding formulations which contain torasemide of the modification I.

The invention relates to a solid pharmaceutical composition which contains torasemide of the modification II or a solvate thereof.

Torasemide (1-isopropyl-3-[(4-m-toluidino-3-pyridyl)sulphonyl]urea) is a loop diuretic which is employed in different doses for the treatment of hypertension, oedema and renal insufficiency.

Torasemide occurs in a number of polymorphic forms. The modifications I and II are described in Acta Cryst., 1978, 2659-2662 and Acta Cryst., 1987,1304-1310. Further polymorphic forms of torasemide and solvates thereof are described in WO 01/10441. The modifications I and II mentioned are designated there as Dupont form 1 and Dupont form 2. A modification III is described in U.S. Pat. No. 6,166,045.

During the preparation and the customary purification, torasemide is always obtained in the form of the modification II. Nevertheless, medicaments which contain the form II are not described in greater detail and are also not on the market. It has specifically been shown that torasemide of the modification II rearranges more or less rapidly to the modification I if it is present in very fine dispersion in a pharmaceutical tablet. Crystal size and rate of dissolution of the active compound can thereby significantly change on introducing the tablet into water, cf. EP 212 537 A (corresponding to U.S. Pat. No. 4,743,693 and U.S. Pat. No. 4,822,807). However, the rate of dissolution is, as is known, one of the most important characteristic variables of a pharmaceutical administration form, for example of a tablet, which must not vary from dose unit to dose unit in order to be able to guarantee reproducible bioavailabilities. The modification I has therefore been employed from the start in torasemide-containing medicaments in order to avoid the disadvantages mentioned. This has the consequence that the preparation of the active compound torasemide of the modification I necessitates a further process step, namely the conversion of the modification II to the modification I, as is described, for example, in EP 212 537 A.

WO 93/00097 describes storage-stable pharmaceutical compositions which contain torasemide and a specific binding agent and a specific disintegrant. However, only the modification I is used.

WO 01/10441 relates to further torasemide modifications. A pharmaceutical composition is also claimed which contains torasemide Dupont form 2, i.e. torasemide of the modification II, and a pharmaceutically acceptable carrier. More detailed particulars for this pharmaceutical composition or for the stability of torasemide of the modification II in this composition are, however, not given. In this respect, the contents of this publication do not thus go beyond the particulars in EP 212 537.

PCT/EP01/02940 describes stable solid or semi-solid pharmaceutical preparations which contain torasemide in essentially non-crystalline form in a solid binder matrix.

The present invention is therefore based on the object of making available stable pharmaceutical compositions which contain torasemide of the modification II and have an active compound release profile which corresponds to that of compositions containing torasemide of the modification I.

Surprisingly, it has now been found that this object is achieved using a solid pharmaceutical composition which contains a high proportion of excipient.

The present invention therefore relates to a solid pharmaceutical composition which comprises

a) torasemide of the modification II or a solvate thereof and

b) at least 50% by weight, based on the total weight of the composition, of at least one excipient which is selected from a sugar, sugar alcohol, calcium hydrogenphosphate, cellulose and cellulose derivatives, and polyvinylpyrrolidones having a molecular weight in the range from 1000 to 15 000.

If not stated otherwise, percentage details in the context of the present invention always mean percentage by weight, based on the total weight of the pharmaceutical composition.

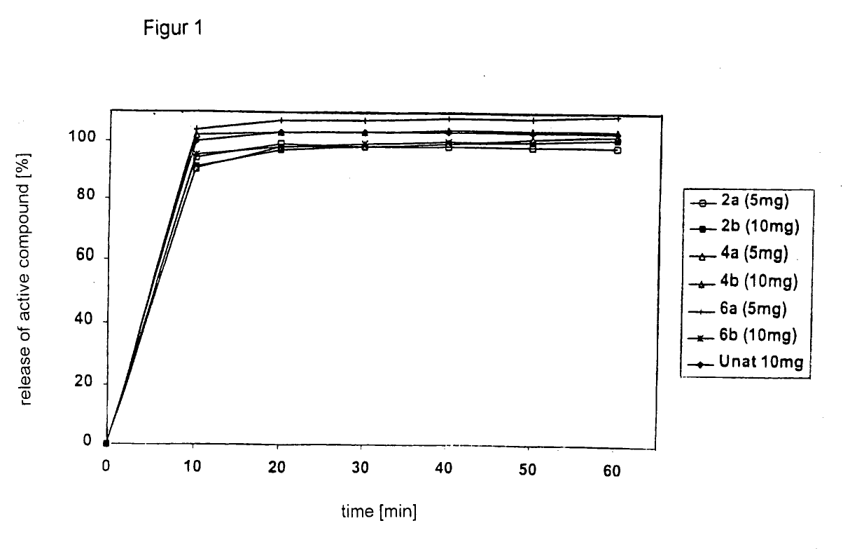

FIG. 1 shows active compound release profiles of tablets according to the invention and of Unat®, a commercial product which contains torasemide of the modification I.

The preparation of torasemide of the modification II is described, for example, in the publications mentioned at the outset in Acta Cryst. and also in DE 25 16 025. Utilizable, pharmaceutically acceptable torasemide salts are salts with organic acids, such as acetic acid, lactic acid, tartaric acid, malic acid, maleic acid, fumaric acid or methanesulphonic acid, and salts with inorganic acids, such as hydrochloric acid, hydrobromic acid, phosphoric acid or sulphuric acid.

Suitable solvates of torasemide of the modification II and their preparation are described in WO 01/10441, to whose contents reference is fully made. The isopropanol solvate is preferred and in particular the ethanol solvate. Their preparation is carried out by suspending torasemide in water and increasing the pH of the mixture by addition of base until the torasemide goes into solution (approximately at pH 9.5 to approximately 10.5). Suitable bases are, for example, ammonium hydroxide, sodium hydroxide, potassium hydroxide, etc. The organic solvent corresponding to the desired solvate is then added, i.e. preferably ethanol or isopropanol, and the pH is lowered by addition of a suitable acid (for example acetic acid, hydrochloric acid, sulphuric acid, etc.) until the solution becomes turbid and the solvate begins to precipitate.

Preferably, the pharmaceutical composition according to the invention contains as component (b) a sugar and/or a sugar alcohol. Sugars and/or sugar alcohols are preferably selected from lactose, glucose, maltose, laevulose, dextrins, mannitol, sorbitol, xylitol and mixtures of two or more thereof. Lactose is particularly preferred.

A particularly suitable component (b) is also microcrystalline cellulose, polyvinylpyrrolidones having a molecular weight in the range from 2000 to 12000 and also cellulose derivatives, in particular hydroxypropylcellulose (e.g. Klucel EF), hydroxypropylmethylcellulose (e.g. Methocel E3 and E5) or methylcellulose or mixtures thereof.

The amount of torasemide contained in the composition according to the invention is in general in the range from 1-15% by weight, preferably 1-10% by weight and in particular 2-5% by weight, in each case based on the total weight of the composition.

The amount of component (b) is preferably at least 50% by weight, particularly preferably at least 60% by weight or at least 70% by weight and in particular at least 80% by weight, in each case based on the total weight of the composition. In general, the composition contains not more than 90% by weight of component (b).

The pharmaceutical composition according to the invention can additionally contain further customary components, such as binding agents, lubricants, disintegrants, wetting agents, etc.

Suitable binding agents which are different from component (b) are, for example, starch and starch derivatives, gum arabic, tragacanth or polyvinylpyrrolidone having a molecular weight of at least 20 000, in particular 20 000 to 1 000 000. The amount of this binding agent is in general in the range from 10-30% by weight, in particular 15-25% by weight.

Suitable disintegrants are, for example, alginic acid, starch, polyacrylic acid, cross-linked polyvinylpyrrolidone, optionally cross-linked carboxymethylcellulose and the salts thereof or optionally cross-linked carboxymethyl starch and the salts thereof. Preferred disintegrants are optionally cross-linked carboxymethylcellulose and optionally cross-linked carboxymethyl starch and their salts, in particular the alkali metal salts, and cross-linked polyvinylpyrrolidone. The amount of disintegrant is in general in the range from 1-10% by weight, in particular 2-8% by weight.

Suitable lubricants are, for example, calcium stearate, magnesium stearate or aluminium stearate, polyethylene glycol, Aerosil (finely disperse silicic acid), stearyl alcohol or cetyl alcohol, talc, siliconized talc, defatted milk powder or sodium stearyl fumarate. Calcium stearate, magnesium stearate or aluminium stearate and Aerosil are preferred. The amount of lubricants is in general in the range from 0.1-3% by weight, in particular 0.2-2% by weight.

Suitable wetting agents are, for example, sodium lauryl sulphate or sodium lauryl ether sulphate. The amount of wetting agent is in general in the range from 0.1 to 1% by weight.

The solid pharmaceutical compositions according to the invention are in general formulated for oral administration and are present, in particular, in the form of tablets, film-coated tablets, powders, pellets, capsules (with a powder or pellet filling).

According to a preferred embodiment, the composition according to the invention is present in the form of a tablet which comprises:

1-10% by weight, preferably 1-5% by weight, of torasemide of the modification II or of a solvate thereof;

50-90% by weight, preferably 60-80% by weight, of component (b);

1-8% by weight of at least one disintegrant; and

optionally 0.1-2% by weight of at least one lubricant.

According to a further preferred embodiment, the composition according to the invention comprises 50-70% by weight of component (b) and 15-25% by weight of at least one binding agent.

A tablet is particularly preferred which comprises:

1-10% by weight, preferably 1-5% by weight, of torasemide of the modification II or of a solvate thereof;

50-70% by weight of component (b); and

15-25% by weight of a binding agent, which is preferably selected from polyvinylpyrrolidone having a molecular weight of at least 20 000, starch and starch derivatives,

1-8% by weight of at least one disintegrant.

The preparation of the composition according to the invention is carried out according to customary pharmaceutical processes. For the preparation of tablets, the components are preferably used with a particle size in the range from 1 μm to 1 mm. In this process, the tablets can be prepared from the powder mixture of the components by direct tableting or after granulation.

The compositions according to the invention are orally administered prophylactically or therapeutically against hypertension and oedema or as a diuretic. The administration of the compositions depends on sex, body weight, age, disease condition, etc. of the patients. Customarily, in the case of an adult a daily dose of approximately 1 to 200 mg, preferably 1-30 mg, of torasemide, divided into 1 to a number of times per day, is administered.

Surprisingly, it has been shown that in the case of the compositions according to the invention containing torasemide of the modification II no rearrangement of the torasemide to the modification I takes place and they thus comply with pharmaceutical quality requirements. On account of the composition, in the case of the preparation of tablets only a low press pressure is necessary and thus a gentle preparation is possible. What is concerned here is a pharmaceutical form of the instant release type, which has a release profile of torasemide which is comparable with the release profile of formulations which contain torasemide of the modification I (see FIG. 1).

The following examples explain the invention without restricting it.

From the constituents indicated below in Table 1, a powder mixture was in each case prepared which was compressed to give tablets on an E1 eccentric press from Fette, stamp diameter 8 mm (biconvex). The information in the table is percentage by weight, based on the total weight of the tablets.

TABLE 1

| Formulation | Ex. 1 | Ex. 2 | Ex. 3 | Ex. 4 | Ex. 5 | Ex. 6 |
|---|---|---|---|---|---|---|
| Torasemide | 3.0 | 3.0 | 3.0 | 3.0 | 3.0 | 3.0 |
| Avicel PH 102 | | | | 20.0 | 20.0 | 20.0 |
| Lactose H₂O | 84.5 | 84.5 | | 69.5 | | 64.5 |
| Methocel K4M | 5.0 | | | | | |
| CaHPO₄ | | | 79.5 | | 69.5 | |
| Kollidon 12 PF | | 5.0 | | | | 5.0 |
| Maize starch | | | 10.0 | | | |
| AcDiSol | 6.0 | | | 3.0 | 3.0 | |
| Kollidon CL | | 6.0 | | | | 3.0 |
| Explotab | | | 6.0 | 3.0 | 3.0 | 3.0 |
| Mg stearate | 0.5 | 0.5 | 0.5 | 0.5 | 0.5 | 0.5 |
| Aerosil 200 | 1.0 | 1.0 | 1.0 | 1.0 | 1.0 | 1.0 |

Avicel PH 102 = microcrystalline cellulose
Methocel K4M = hydroxypropylmethylcellulose (molecular weight approximately 40 000;. Ph. Eur.)
Kollidon 12 PF = polyvinylpyrrolidone having a molecular weight of approximately 2500 and of pharmaceutical quality (Ph. Eur.)
AcDiSol = cross-linked carboxymethylcellulose
Kollidon CL = cross-linked polyvinylpyrrolidone
Explotab = sodium carboxymethyl starch
Aerosil 200 = pure silicic acid (Ph. Eur.)

All excipients were employed in pharmacopoeia quality.

On the basis of the recipes of Examples 1 to 6 and according to the process indicated above, tablets containing 5 mg and 10 mg of torasemide of the modification II were in each case prepared. The breaking strength, the friability, the disintegration time of these tablets and the active compound release profile of the tablets of Examples 2, 4 and 6 were determined in comparison to the commercial product Unat®. Breaking strength and friability were determined as described in Sucker, Fuchs, Speiser, Pharmazeutische Technologie, Thieme Verlag Stuttgart, 2nd Edition, 1991, page 330, 331. The active compound release profile was determined according to the paddle model of USP 24. The disintegration time was determined according to Ph. Eur. The results are shown in Table 2 and FIG. 1 below.

TABLE 2

| Formulation | Example 1 | | Example 2 | | Example 3 | | Example 4 | | Example 5 | | Example 6 | |
|---|---|---|---|---|---|---|---|---|---|---|---|---|
| Dose | 5 mg | 10 mg | 5 mg | 10 mg | 5 mg | 10 mg | 5 mg | 10 mg | 5 mg | 10 mg | 5 mg | 10 mg |
| Average mass [mg] | 168.4 | 329.7 | 165.4 | 328.6 | 167.5 | 333.5 | 167.8 | 332.2 | 167.3 | 335.1 | 167.1 | 338.4 |
| ± | ± | ± | ± | ± | ± | ± | ± | ± | ± | ± | ± | ± |
| sdvᵣₑₗ (n = 100) | 1.1% | 1.1% | 0.5% | 0.6% | 0.5% | 0.6% | 0.4% | 0.7% | 0.5% | 0.6% | 0.8% | 0.7% |
| Breaking strength | 43 | 52 | 48 | 54 | 46 | 59 | 55 | 63 | 37 | 63 | 50 | 51 |
| [N] (n = 10) | | | | | | | | | | | | |
| Friability [%] | n.d. | 0.4 | 0.2 | 0.3 | 0.5 | 0.8 | 0.1 | 0.7 | 0.2 | 0.8 | 0.1 | 0.4 |
| (n = 20) | | | | | | | | | | | | |
| Disintegration | n.d. | n.d. | 15 | 30 | 12 | 12 | 14 | 30 | 9 | 21 | 20 | 24 |
| [sec] | | | | | | | | | | | | |
| in H₂O, 37° C. | | | | | | | | | | | | |
| (n = 6) | | | | | | | | | | | | |

It can be seen that the tablets satisfy the pharmaceutical quality requirements. It can moreover be seen from FIG. 1 that the tablets according to the invention have an active compound release profile comparable to the commercial product Unat, which contains 10 mg of torasemide of the modification I per tablet.

1. Solid pharmaceutical composition, comprising
a) torasemide of the modification II or a solvate thereof and
b) at least 50% by weight, based on the total weight of the composition, of at least one excipient which is selected from a sugar, sugar alcohol, calcium hydrogenphosphate, cellulose, cellulose derivatives and polyvinylpyrrolidones having a molecular weight in the range from 1000 to 15 000.

2. Composition according to claim 1, which comprises at least 60% by weight of component (b).

3. Composition according to claim 1, where the sugar or sugar alcohol is selected from lactose, glucose, maltose, laevulose, dextrins, mannitol, sorbitol, xylitol and mixtures of two or more thereof.

4. Composition according to claim 1, where component (b) is selected from lactose, calcium hydrogenphosphate, microcrystalline cellulose, hydroxypropylmethylcellulose, hydroxypropylcellulose, methylcellulose, polyvinylpyrrolidone having a molecular weight in the range from 2000 to 12 000 and mixtures thereof.

5. Composition according to claim 1, which comprises 1-15% by weight of torasemide of the modification II.

6. Composition according to claim 1, which additionally comprises at least one binding agent, lubricant and/or disintegrant.

7. Composition according to claim 1, comprising 50-70% by weight of component (b) and 15-25% by weight of a binding agent which is different from component (b).

8. Composition according to claim 1 in the form of a tablet.

9. Composition according to claim 8, comprising:
1-10% by weight of torasemide of the modification II or of a solvate thereof,
50-80% by weight of component (b) and
1-8% by weight of at least one disintegrant.

10. Composition according to claim 9, comprising:
1-10% by weight of torasemide of the modification II or of a solvate thereof,
60-90% by weight of component (b) and
1-8% by weight of at least one disintegrant.

11. Composition according to claim 9, comprising:
1-10% by weight of torasemide of the modification II or of a solvate thereof,
50-70% by weight of component (b),
15-25% by weight of a binding agent which is selected from polyvinylpyrrolidone having a molecular weight of at least 20 000, starch and starch derivatives, and
1-8% by weight of at least one disintegrant.

12. Composition according to claim 9, where the disintegrant is selected from optionally cross-linked carboxymethylcellulose and the salts thereof, optionally cross-linked carboxymethyl starch and the salts thereof, cross-linked polyvinylpyrrolidone and mixtures thereof.
